(19) United States
(12) Patent Application Publication
(10) Pub. No.: US 2024/0156624 A1
(43) Pub. Date: May 16, 2024

(54) HYBRID STENT AND STENT RETRIEVER

(71) Applicant: KAI MEDTECH, LLC, LAKE FOREST, CA (US)

(72) Inventors: LINH A. DINH, IRVINE, CA (US); JOSHUA A. BENJAMIN, MISSION VIEJO, CA (US)

(73) Assignee: KAI MEDTECH, LLC, LAKE FOREST, CA (US)

(21) Appl. No.: 17/781,649

(22) PCT Filed: Jan. 5, 2022
(86) PCT No.: PCT/US2022/011250

§ 371 Date: Jun. 1, 2022

Related U.S. Application Data
(60) Provisional application No. 63/273,409, filed on Oct. 29, 2021.
(60) Provisional application No. 63/139,684, filed on Jan. 20, 2021.

(51) Int. Cl. A61F 2/915; A61F 2/07; A61F 2/95; A61F 2/966; A61L 31/04

(52) U.S. Cl. CPC A61F 2/915; A61F 2/07; A61F 2/966; A61L 31/04; A61F 2002/91541; A61F 2002/91575; A61F 2002/9528; A61F 2210/0014; A61F 2220/0025; A61F 2230/0069; A61F 2310/00023

(57) ABSTRACT

A stent including a hybrid network cluster of open cells and closed cells arranged wherein said closed cells are connected in a configuration to be resheathable. Open cells are not connected and the stent can be unsheathed to enhance flexibility. In one embodiment a ring of proximal closed cells are tapered and the stent is retrievable by engagement of a pushwire assembly.

### CROSS-REFERENCE TO RELATED APPLICATION

This application is a national stage application, filed under 35 U.S.C. § 371, of International Patent Application No. PCT/US2022/011250 filed Jan. 5, 2022 which claims the benefit of U.S. Provisional Application No. 63/139,684, entitled “HYBRID STENT” filed Jan. 20, 2021 and U.S. Provisiona Application No. 63/273,409 entitled “HYBRID STENT AND STENT RETRIEVER” filed Oct. 29, 2021. International Patent Application No. PCT/US2022/011250 and U.S. Provisional Applications Nos. 63/139,684 and 63/273,409 are incorporated by reference herein in their entireties.

### BACKGROUND OF THE INVENTION

### 1. Field of the Invention

The present invention relates generally to the field of endovascular treatment of blood vessels, and more particularly to stent devices and systems. In some embodiments, these stent devices and systems relate to hemodynamically significant intracranial atherosclerotic disease (ICAD) and Acute Ishemic Stroke (AIS).

### 2. Description of the Related Art

Medical devices that can benefit from the present invention include those that are characterized by hollow interiors and that are introduced endoluminally and expand when deployed. These are devices that move or are moved between collapsed and expanded conditions or configurations for ease of deployment through catheters and introducers. Such devices are typically introduced to a diseased location within a body vessel (e.g., a stenosed section or an aneurysm) and may perform a variety of functions, including support and/or occlusion.

Endoluminal stents typically have a relatively open structure, with a plurality of interconnecting struts which define pores or openings in and/or through the surface that can allow for endothelialization and more permanent fixture of the stent within the vessel after implantation. Certain stents have an especially open structure in order to allow blood flow through the openings and to peripheral arteries after implantation of the stent adjacent to an aneurysm. Typically, the pores or openings are added by masking and/or etching techniques or laser- or water-jet cutting. Known stents include the Cordis Enterprise™ line of self-expanding stents, which are described in numerous patents and published patent applications, including U.S. Pat. Nos. 6,612,012; 6,673,106; 6,818,013; 6,833,003; 6,955,685; 6,960,227; 7,001,422; and 7,037,331 and U.S. Patent Application Publication No. 2005/0234536, all of which are hereby incorporated by reference hereinto.

One potential drawback of known stents is that they may incorporate relatively complicated strut or cell structures that may prohibit easy manipulation of the design, such as when the diameter of the stent is changed. For example, from a manufacturing perspective, a stent design may have cell shapes and characteristics that are well suited to achieve desired effects or operational characteristics when manufactured at a given nominal size or diameter, but these shapes or characteristics may have to be changed or adjusted to maintain identical operational characteristics for a stent manufactured with a different nominal size or diameter. Further, when the struts and/or cells are formed using a laser- or watercutting process, a complicated pattern may require a high degree of cutting time.

Accordingly, there is a need for an approach to provide stents having an improved cell structure, particularly one that incorporates relatively uncomplicated cell structures and that accommodates manufacture of stents of differing nominal sizes without having to redesign cells during manufacturing. A need remains for a stent cell scheme that facilitates achieving desired hemodynamics in the body vessel and the chronic outward force and radial resistive force of the stent needed for a variety of nominal sizes through variations with cells of identical shapes.

There is a need for an approach to provide stents with improved cell structures providing resheathing of the stent. In partial response to this problem Tenne (U.S. Pat. No. 8,062,347 B2) discloses a resheathable stent; however, it is relatively rigid. As will be disclosed below the present invention provides a resheathable stent with enhanced flexibility.

Also, there is a need for an approach to provide temporary stents, also known as “stentretrievers” or “stentrievers”, due to acute ischemic stroke to provide immediate blood flow restoration to a vessel occluded by a clot and, after reestablishing blood flow, address the clot itself. In a response to this problem Ferrera et al. (U.S. Pat. No. 8,574,262 B2) provide a potential solution to immediate blood flow restoration. The invention can advantageously facilitate natural lysis of the clot and also reduce or obviate the concern for distal embolization due to fragmentation of the clot. Several embodiments of the invention are disclosed that provide for progressive, or modular, treatment based upon the nature of the clot. The stent described in Ferrera et al. is a closed cell design and does not conform to the vessel shapes adequately.

Furthermore, Ulm, Ill et al. (U.S. Pat. No. 10,888,346 B2) provide a platform of devices for removing obstructions and other objects within a blood vessel or other interior lumen of an animal. The system may be deployed in the lumen from a catheter(s) and may include a strain gauge for measuring tension on the pull wire. A number of different baskets designs are disclosed in the invention. Methods of manufacturing such baskets out of a single tube of a memory metal without the need for any welding, and methods of use are also disclosed. The design structure described in this patent does not provide adequate pushability to the target lesion, due to limited amount of connecting links.

### SUMMARY OF THE INVENTION

In one aspect, the present invention is embodied as a stent including a hybrid network cluster of open cells and closed cells arranged wherein said closed cells are connected in a configuration to be resheathable. Open cells are not connected and the stent can be unsheathed to enhance flexibility.

In a preferred embodiment the hybrid network cluster of open cells and closed cells includes a plurality of rings of the closed cells. Each ring of the closed cells, includes:
a) a plurality of pairs of closed cells;
b) a plurality of straight connecting elements, each straight connecting element connecting a pair of closed cells to an adjacent pair of circumferentially spaced closed cells; and,
c) a plurality of flexible connecting elements, each flexible connecting element connecting longitudinally adjacent rings, wherein each pair of closed cells comprises a proximal peak at a proximal end and a distal peak at a distal end, said proximal peaks of a ring being connected by a flexible connecting element to a valley of an adjacent spaced ring.

The distal peaks are free from constraint to enhance flexibility and the proximal peaks are constrained for resheathability.

In another aspect, the present invention is embodied as a stent delivery system, including:
a) a catheter;
b) a shaft comprising a distal end, disposed within the catheter; and
c) a stent including a hybrid network cluster of open cells and closed cells arranged wherein said closed cells are connected in a configuration to be resheathable. Open cells are not connected and the stent can be unsheathed to enhance flexibility.

In another aspect, the present invention is embodied as a method for deploying a resheathable stent for stent assisted coiling of hemorrhagic aneurysms and for treatment of intracranial atherosclerotic disease. This method includes inserting a catheter into a vasculature of a patient wherein a resheathable stent system is disposed with the catheter. The resheathable expandable stent includes a hybrid network cluster of open cells and closed cells arranged wherein the closed cells are connected in a configuration to be resheathable; and, open cells are not connected and the stent can be unsheathed to enhance flexibility. Longitudinal movement of the shaft relative to the resheathable expandable stent expands and contracts the resheathable stent.

In another aspect, the present invention is embodied as a method for deploying a resheathable, yet temporary stent for stent assisted coiling of hemorrhagic aneurysms and for treatment of intracranial atherosclerotic disease.

In another aspect the stent is embodied as a retrievable stent comprising having a proximal end and a distal end, wherein the hybrid network cluster of open cells and closed cells comprises a plurality of rings of the closed cells, each ring of closed cells comprising: a) a plurality of closed cells; b) a plurality of distally directed connecting elements, each distally directed connecting element connecting a closed cell of said plurality of closed cells to an adjacent circumferentially spaced closed cell via an associated distally directed connecting element of said adjacent circumferentially spaced closed cell; c) a plurality of proximally directed connecting elements, each proximally directed connecting element connecting longitudinally adjacent rings, wherein each closed cell comprises a distal peak and a proximal peak, said proximal peak of said closed cell being connected by a proximally directed connecting element to a valley of an adjacent spaced ring; a first proximal ring being tapered; and, d) a pushwire assembly positionable within an introducer sheath of a stent delivery system, said pushwire having a proximal pushwire end and a distal pushwire end, said distal pushwire end being attached to said proximal peaks of closed cells of said first proximal ring.

In another broad aspect of the retrievable stent, each ring comprises:
a) a plurality of closed cells, each closed cell, comprising: i) a substantially diamond-like shape structure, including: 1. a first cell strut; 2. a second cell strut opposing said first cell strut; 3. a third cell strut connecting said first cell strut to said second cell strut at a distal peak at an end of the ring; and, 4. a fourth cell strut connecting said second cell strut to said first cell strut; and,
b) a plurality of first distally directed connecting elements, each first distally directed connecting elements connecting a distal apex of an open cell to an adjacent closed cell, said plurality of first distally directed connecting elements comprising a set of distally directed connecting elements associated with each of said closed cells, wherein each set of distally connecting elements, comprises: i. a first distally directed connecting element extending from said distal apex of an open cell, said distal apex of an open cell being at a connection point of a second distally directed connecting element and said adjacent closed cell; ii. a second distally directed connecting element extending from said distal apex of an open cell, said distal apex of an open cell being at a connection point of said first distally directed connecting element and said adjacent closed cell; and wherein said first distally directed connecting element functions as a second distally directed connecting element to a distal apex of an adjacent circumferentially spaced closed cell;
c) a plurality of proximally directed connecting elements, each proximally directed connecting element connecting longitudinally adjacent rings, said plurality of proximally directed connecting elements comprises a set of proximally directed connecting elements associated with each said closed cell and a valley of an adjacent ring, wherein each set of proximally directed connecting elements, comprises:
i. a first proximally directed connecting element extending from a proximal peak of said closed cell; and,
ii. a second proximally directed connecting element extending from said valley; wherein the distal peaks are free from constraint to enhance flexibility and wherein the proximal peaks are constrained for resheathability; and,
d) a pushwire/pusher assembly positionable within an introducer sheath of a stent delivery system, said pusher assembly having a proximal pushwire end and a distal pushwire end, said distal pushwire end being attached to proximal peaks of closed cells of said first proximal ring.

In another broad aspect, the present invention is embodied as a retrievable stent including a hybrid network cluster of open cells and closed cells arranged wherein said closed cells are connected in a configuration to be resheathable; and, wherein open cells are not connected and the stent can be unsheathed to enhance flexibility; and, wherein a ring of proximal closed cells are tapered and the stent is retrievable.

### BRIEF DESCRIPTION OF THE DRAWINGS

FIG. 1 is a perspective illustration of a first embodiment of the stent of the present invention.

FIG. 2 is a side elevation of the embodiment of FIG. 1.

FIG. 3 is a flattened pattern of the embodiment of FIG. 1.

FIG. 4 is a flattened pattern of the embodiment of FIG. 1, with a ring being isolated and remainder of the stent shown in phantom.

FIG. 5 is an enlarged detail of a pair of closed cells and its associated straight connecting elements and flexible connecting elements.

FIG. 6 is a perspective view of the enlarged detail of FIG. 5.

FIG. 7 is a perspective illustration of a second embodiment of the stent of the present invention.

FIG. 8 is a flattened pattern of the embodiment of FIG. 7.

FIG. 9 illustrates a stent in an expanded configuration relative to a stent delivery system.

FIG. 10 shows the stent being resheathed.

FIG. 11 is a perspective illustration of a third embodiment of the stent of the present invention.

FIG. 12 is a side elevation of the embodiment of FIG. 11.

FIG. 13 is a flattened pattern of the embodiment of FIG. 11.

FIG. 14 is a flattened pattern of the embodiment of FIG. 11, with a ring being isolated and remainder of the stent shown in phantom.

FIG. 15 is an enlarged detail of a closed cell and its associated connecting elements.

FIG. 16 is a perspective view of the enlarged detail of FIG. 15.

FIG. 17 is a perspective illustration of a fourth embodiment of the stent of the present invention.

FIG. 18 is a side elevation of the embodiment of FIG. 17.

FIG. 19 is a flattened pattern of the embodiment of FIG. 17.

FIG. 20 is a flattened pattern of the embodiment of FIG. 17, with a ring being isolated and remainder of the stent shown in phantom.

FIG. 21 is an enlarged detail of a closed cell and its associated connecting elements.

FIG. 22 is a perspective view of the enlarged detail of FIG. 17.

The same elements or parts throughout the figures of the drawings are designated by the same reference characters, while equivalent elements bear a prime designation.

### DETAILED DESCRIPTION OF THE INVENTION

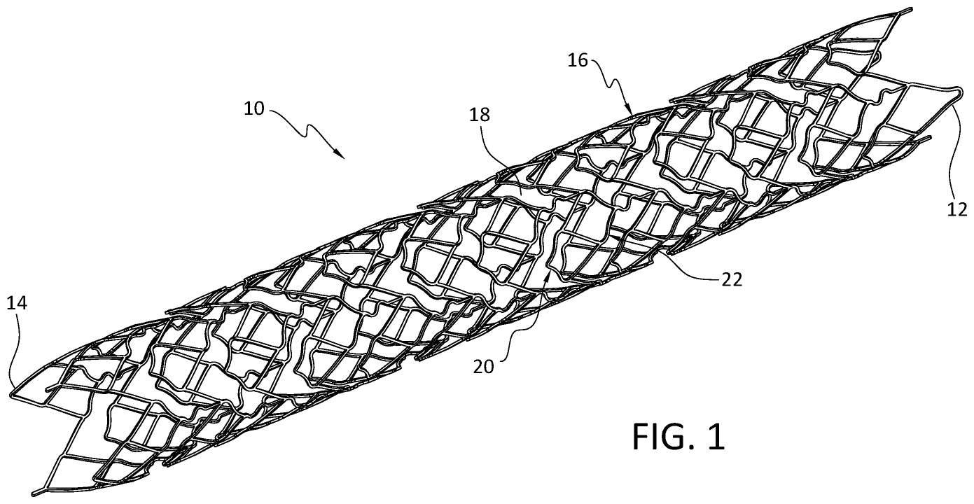

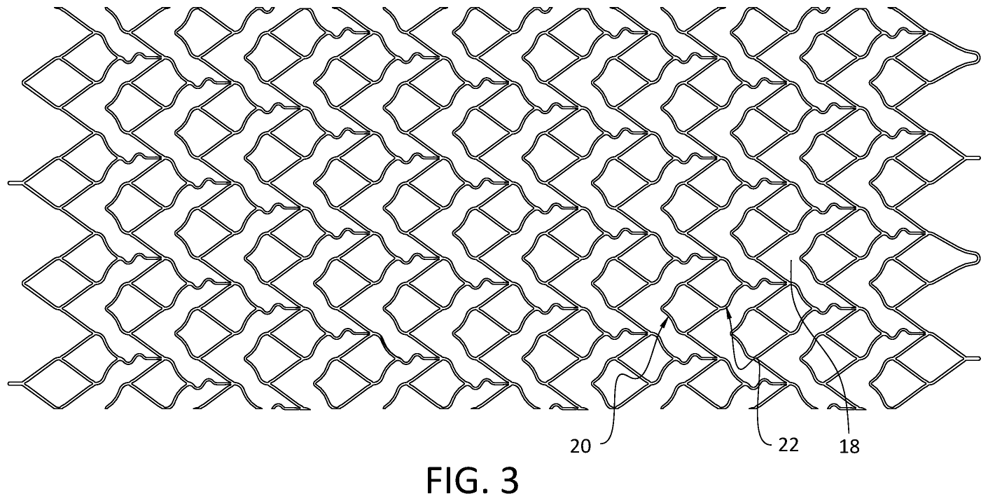

Referring now to the drawings and the characters of reference marked thereon, FIGS. 1-5 show a first embodiment of the stent implantable within a body vessel of a subject, designated generally as 10. The stent 10 has a proximal end 12 and a distal end 14 as oriented relative to the manner in which it is introduced. It includes a hybrid network cluster 16 of open cells 18, distal closed cells 20, and proximal closed cells 22. As will be disclosed below, the closed cells 20, 22 are connected in a configuration to be resheathable. The open cells 18 are not connected and the stent can be unsheathed to enhance flexibility.

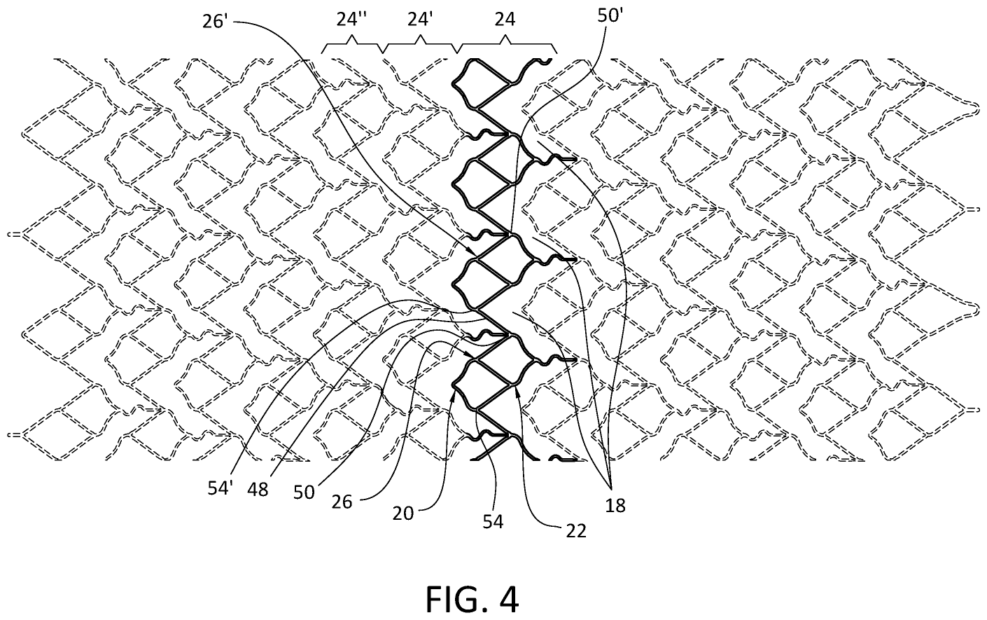

As can be best be seen in FIG. 4, the hybrid network cluster of open cells and closed cells comprises a plurality of rings 24, 24′, 24″ of pairs of the closed cells 20, 22. Each pair, designated generally as 26, includes a distal closed cell 20 and a proximal closed cell 22.

Each distal closed cell 20 has a substantially diamond-like shape. Each distal closed cell 20 includes a first distal cell strut 28, a second distal cell strut 30 opposing the first distal cell strut 28, a third distal cell strut 32 connecting the first distal cell strut 28 to the second distal cell strut 30 at a distal peak 34 at a distal end of the ring 24, and a shared strut 36 connecting the first distal cell strut 28 to the second distal cell strut 30.

The struts may have strut wall thicknesses in a range of 0.05-0.15 mm, preferably about 0.076 mm. The strut widths are approximately the same.

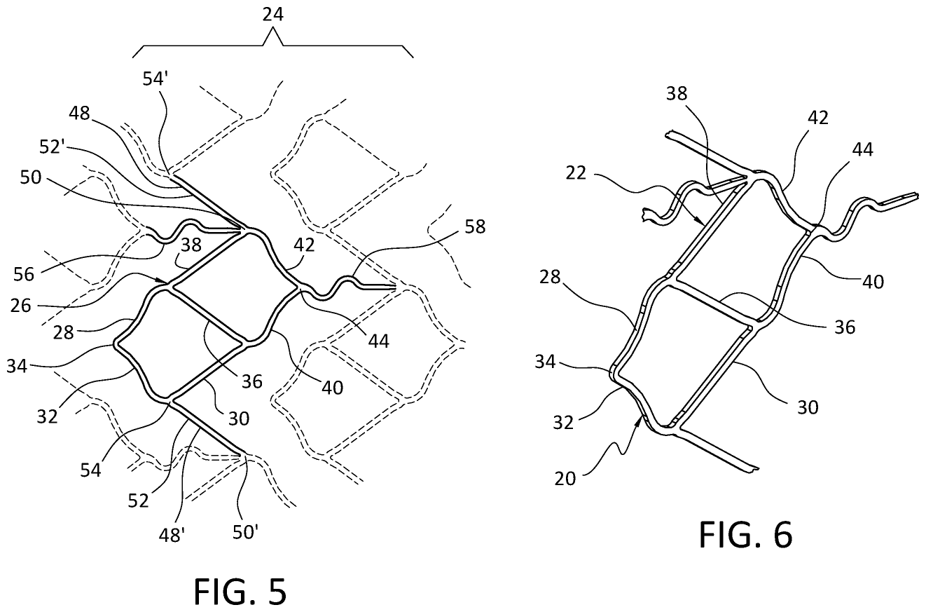

As best seen in FIGS. 5-6, each proximal closed cell 22 has a substantially diamond-like shape. Each proximal closed cell 22 includes a first proximal cell strut 38, a second proximal cell strut 40 opposing the first proximal cell strut 38, a third proximal cell strut 42 connecting the first proximal cell strut 38 to the second proximal cell strut 40 at a proximal peak 44 at a proximal end of the ring 24, and the shared strut 36 connecting the first proximal cell strut 38 to the second proximal cell strut 40.

Each ring 24 includes a plurality of straight connecting elements. Each straight connecting element connects a pair of closed cells to an adjacent pair of circumferentially spaced closed cells. A set of straight connecting elements is associated with each pair of said closed cells. With reference to pair 26 of closed cells, the set of straight connecting elements includes a first straight connecting element 48 extending from a first apex 50. The first apex 50 is a connection point of the first proximal cell strut 38 and the third proximal cell strut 42. A second straight connecting element 52 extends from a second apex 54. The second apex 54 is at a connection point of the second distal cell strut 30 and the third distal cell strut 32.

The first straight connecting element 48 functions as the second straight connecting element 52′ to a first apex 50′ of an adjacent second apex 54′ of an adjacent pair 26′ of circumferentially spaced closed cells.

Each ring includes a plurality of flexible connecting elements. Each flexible connecting element connects longitudinally adjacent rings. A set of flexible connecting elements is associated with each pair of the closed cells. With reference to pair 26 of closed cells, the set of flexible connecting elements includes a first flexible connecting element 56 extending from the first apex 50. A second flexible connecting element 58 extends from the proximal peak 44.

The first flexible connecting element 56 functions as a second flexible connecting element 58′ to a first apex 50′ of an adjacent pair 26′ of longitudinally spaced closed cells of an adjacent ring 24′. The first apex is positioned at a valley of the adjacent longitudinally spaced ring. In a preferred embodiment each flexible connecting element comprises a straight or an “S” configuration. There may be other suitable type of flexible connecting elements, such as, that may have a “V” configuration.

Thus, the distal peaks are free from constraint to enhance flexibility and wherein the proximal peaks are constrained for resheathability. This allows the stent to have the advantages of closed cell systems, i.e. radial strength to open plaques, etc. At the same time it has the advantages of open cells systems, i.e. flexibility. Thus, the present invention is very advantageous for ICAD applications.

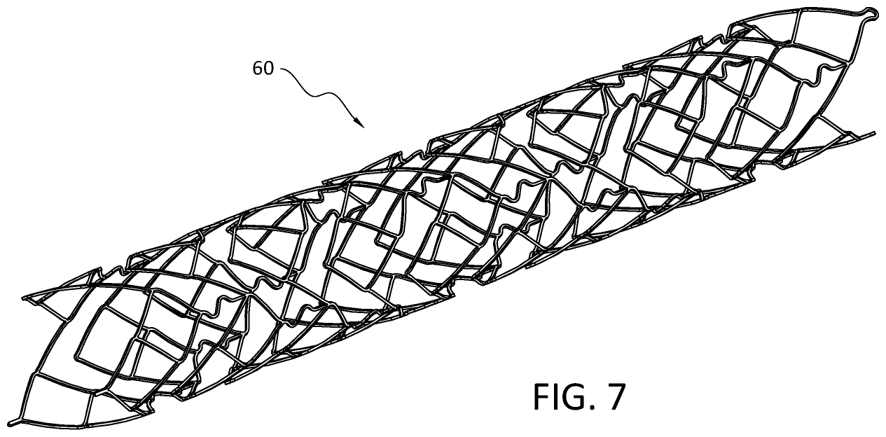

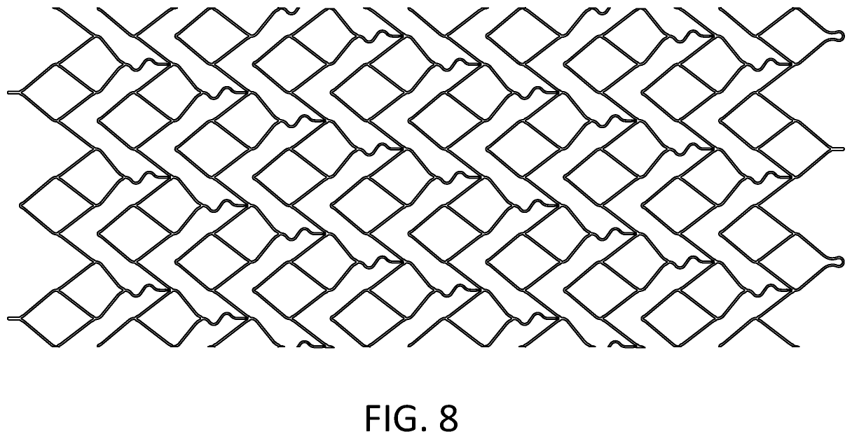

The stent 10 illustrated in FIGS. 1-4 has rings that comprise 4 pairs of closed cells. Thus, it is an eight crown device. In FIGS. 7-8 a second embodiment of a stent, illustrated generally as 60, in which each ring has 3 pairs of closed cells. Thus, it is a 6 crown device. Generally speaking, the stent may have two to ten pairs of closed cells.

The hybrid network cluster of open cells and closed cells has a diameter in a range of preferably about 2 mm to 12 mm in a fully open position and a length in a range of preferably about 10 mm to 60 mm in a fully open position.

A preferred utilization of this embodiment of the stent, i.e. stent 10, is in neurovascular anatomy for acute ischemic strokestent assisted coiling of hemorrhagic aneurysms and for treatment of intracranial atherosclerotic disease.

The stent is preferably formed of a shape memory alloy (SMA), such as nitinol. Alternatively, the stent may be formed of stainless steel, cobalt chromium, bioresorbable plastics or other suitable metals.

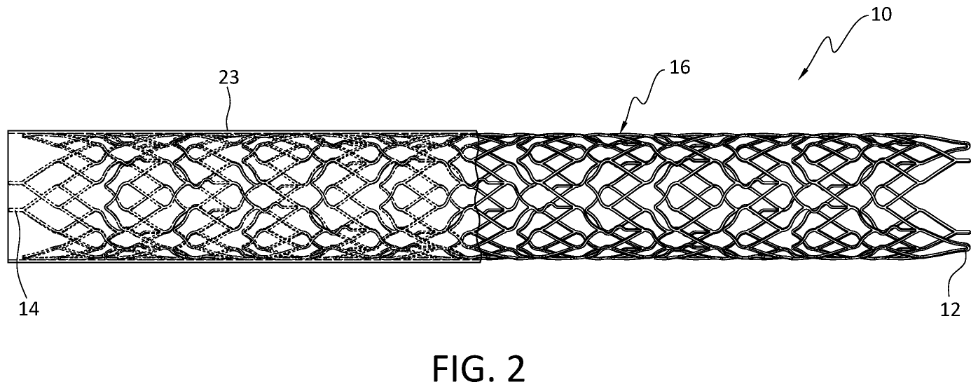

Referring to FIG. 2, in some embodiments the stent further includes a graft 23 formed of expanded polytetrafluoroethylene (ePTFE) material positioned on the outer surface of the hybrid network cluster of open cells and closed cells.

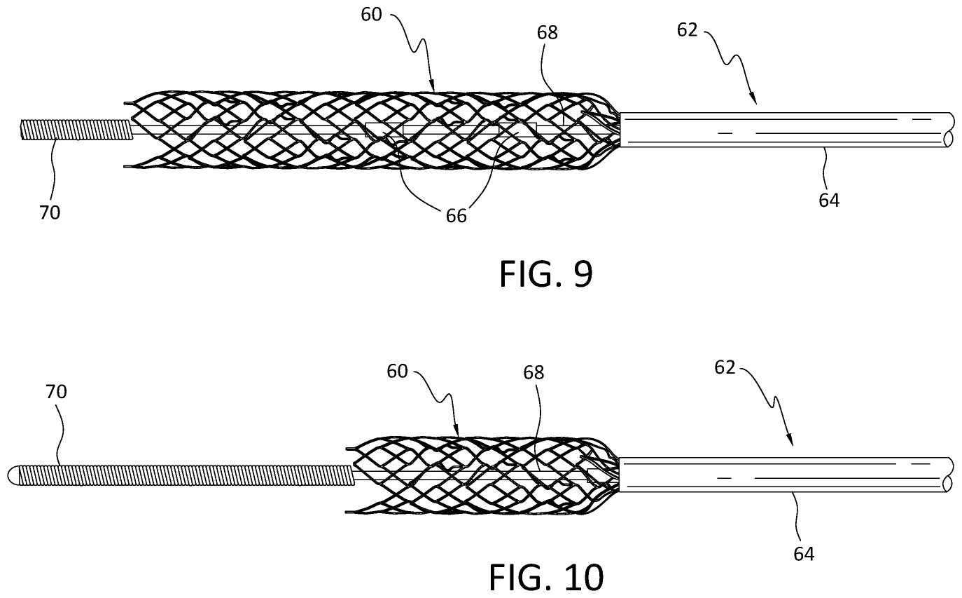

FIG. 9 shows a stent 60 (of the FIGS. 7-8 type) positioned relative to a stent delivery device, designated generally as 62. The stent delivery device may include components well known including an introducer sheath 64 (i.e. catheter), marker bands 66, a core wire 68, a distal tip coil 70, and a proximal coil (hidden by the introducer sheath).

The cell design may be manufactured by a number of methods, such as laser cutting, etc.

In some embodiments, the expandable stent may be drug-eluting. The expandable stent may include a drug covering or coating selected from the group of Everolimus, Paclitaxel, Siromlimus, Corolimus and any other related compounds, salts, moieties which potentially reduce the risk of thrombosis, lumen loss, and related challenges. In some embodiments, the expandable stent may include radiopaque markers, such as platinum, gold, silver, or tantalum. In some embodiments, the expandable stent may be fabricated from bioabsorbable materials, such as magnesium based materials, polylactic acid-based (PLA's) polymers, and the like.

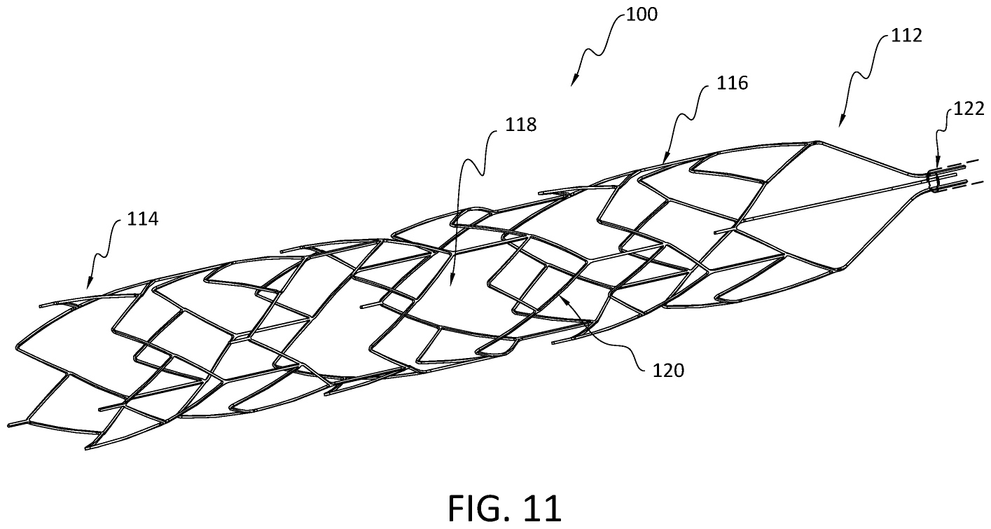

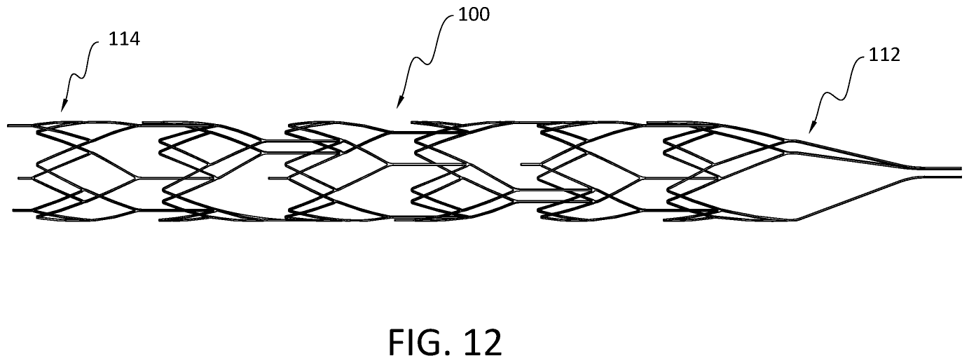

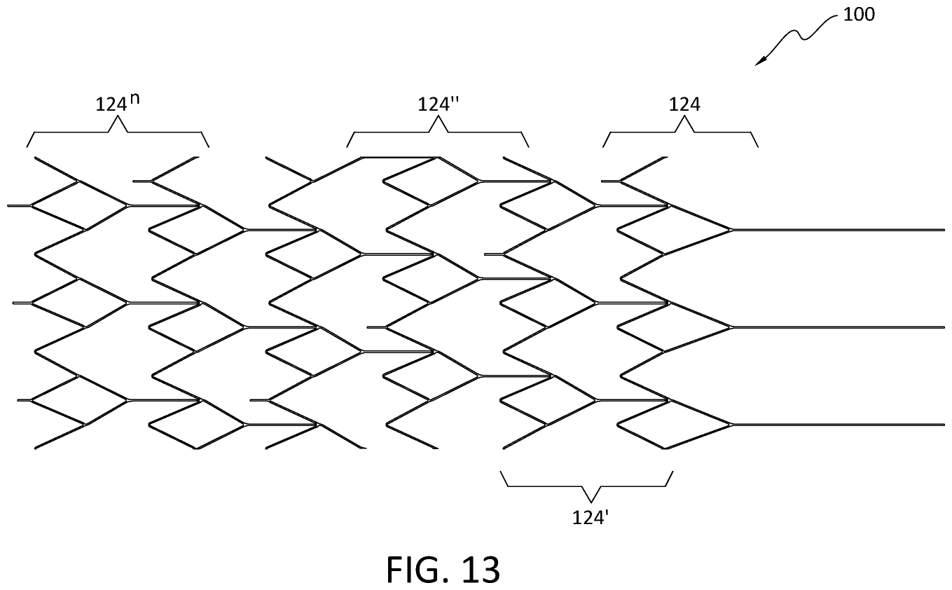

Referring now to FIG. 11-16, a third embodiment of a stent in accordance with the principles of the present invention is illustrated, designated generally as 100, in which a stent is provided that is retrievable and temporary. As in the previous embodiments, the stent 100 has a proximal end 112 and a distal end 114 as oriented relative to the manner in which it is introduced. It includes a hybrid network cluster 116 of open cells 118, and closed cells 120. The open cells 118 are not connected and the stent can be unsheathed to enhance flexibility. Further, as will be disclosed below, beyond the capabilities of the first two embodiments, in this third embodiment, the closed cells 120 are connected in a configuration to be retrievable. This capability is enabled by a pushwire assembly 122.

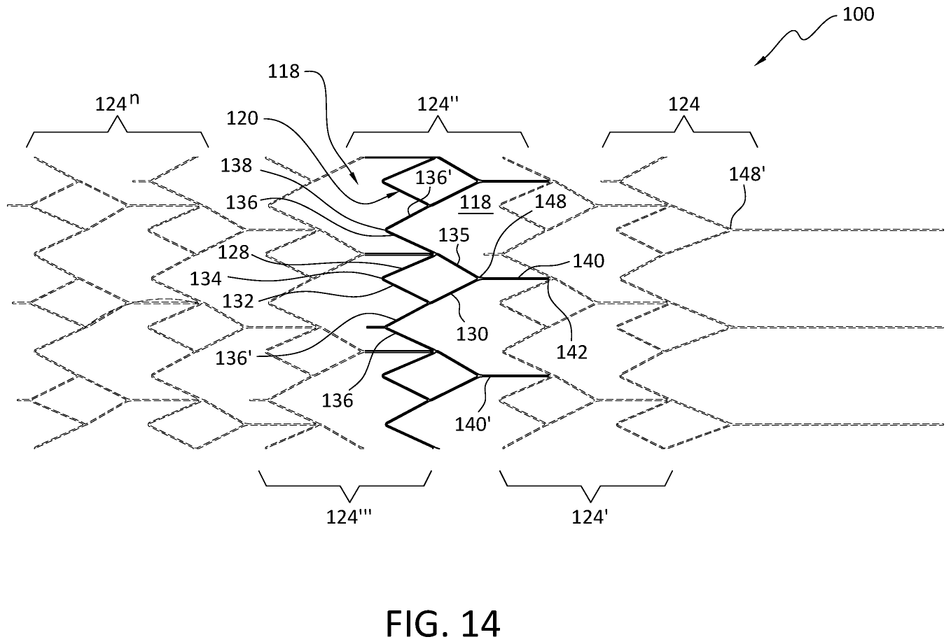

As can be seen in FIG. 14, the hybrid network cluster of open cells and closed cells comprises a plurality of rings 124, 124′, 124″ . . . 124ⁿ of the closed cells 120. Each closed cell 120 has a substantially diamond-like shape structure. Each closed cell 120 includes a first cell strut 128; a second cell strut 130 opposing the first cell strut 128; a third cell strut 132 connecting the first cell strut 128 at a distal peak 134 at an end of the respective ring 124, 124′, 124″; and, a fourth cell strut 135 connecting the second cell strut 130 to the first cell strut 128.

Each ring 124, 124′, 124″ . . . 124ⁿ includes a first distally directed connecting element 136. Each first distally directed connecting element 136 connects a distal apex 138 of an open cell 118 to an adjacent closed cell 120. The plurality of first distally directed connecting elements 136 includes a set of the distally directed connecting elements (i.e. 136, 136′) associated with each of the closed cells 120.

Reiterating, each set 136, 136′ of distally connecting elements includes a first distally directed connecting element 136 extending from the distal apex 138 of an open cell 118, the distal apex 138 being at a connection point of a second distally directed connecting element 136′ and the adjacent closed cell 120.

The second distally directed connecting element 136′ extends from the distal apex 138 of an open cell 118, the distal apex 138 of an open cell 118 being at a connection point of the first distally directed connecting element 136 and the adjacent closed cell 120.

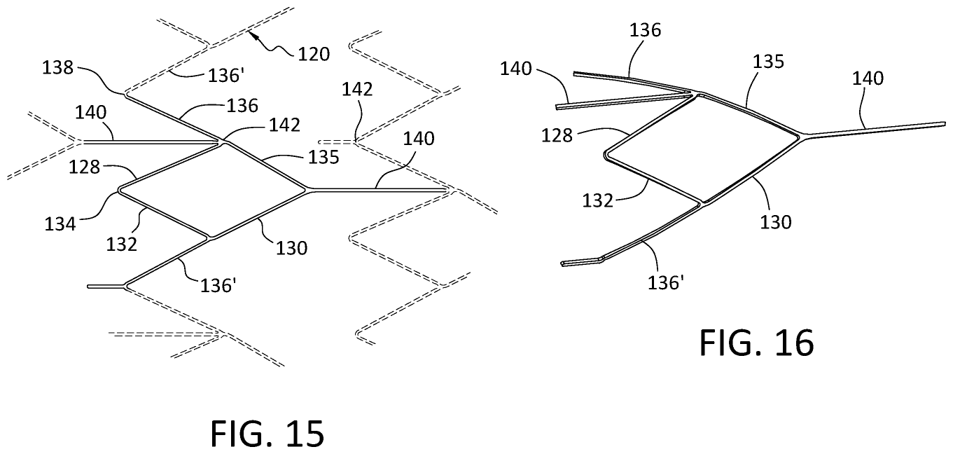

Thus, as can perhaps best be seen in FIG. 15, the first distally directed connecting element 136 functions as a second distally directed connecting element 136′ to the distal apex 138 of an adjacent circumferentially spaced closed cell 120.

Each ring 124, 124′, 124″ . . . 124ⁿ includes a plurality of proximally directed connecting elements 140, 140′, each proximally directed connecting element 140, 140′ connecting longitudinally adjacent rings 124. The plurality of proximally directed connecting elements includes a set of proximally directed connecting elements 140, 140′ associated with each closed cell 120 and a valley 142 of an adjacent ring 124. Each set of proximally directed connecting elements includes a first proximally directed connecting element 140 extending from a proximal peak 148 of the closed cell; and, a second proximally directed connecting element 140′ extending from the valley 142.

Thus, the first proximally directed connecting element 140 functions as a second proximally directed connecting element to the valley 142 of an adjacent ring, wherein the valley is positioned at an apex of an open cell of an adjacent longitudinally spaced ring.

A pushwire assembly 144 is positionable within an introducer sheath of a stent delivery system (discussed above relative to FIG. 9). The pushwire assembly has a proximal pushwire end (not shown) and a distal pushwire end 146. The distal pushwire end 146 is attached to proximal peaks 148′ of closed cells 120 of the first proximal ring 124. The first proximal ring 124 is tapered toward the distal pushwire end 146. It is preferably welded to the distal pushwire end 146. The pushwire assembly 144 preferably includes a flexible laser cut hypotube formed of stainless steel with a core wire formed of Nitinol® alloy.

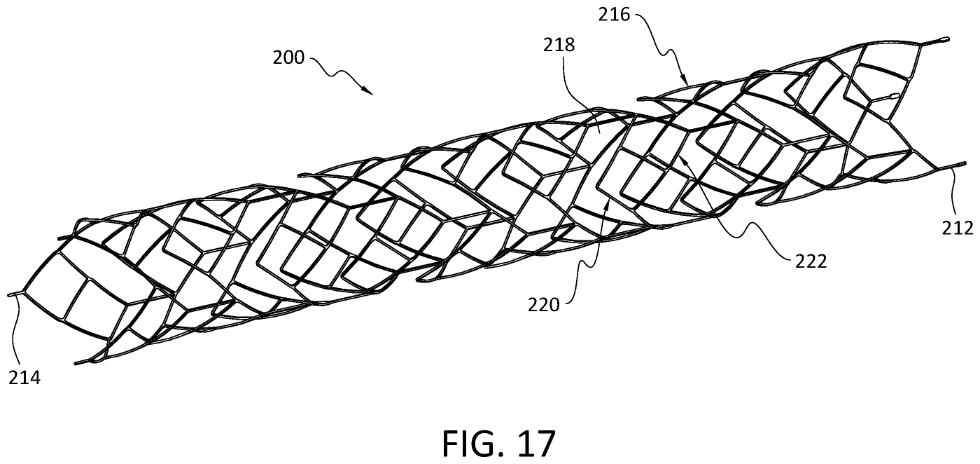

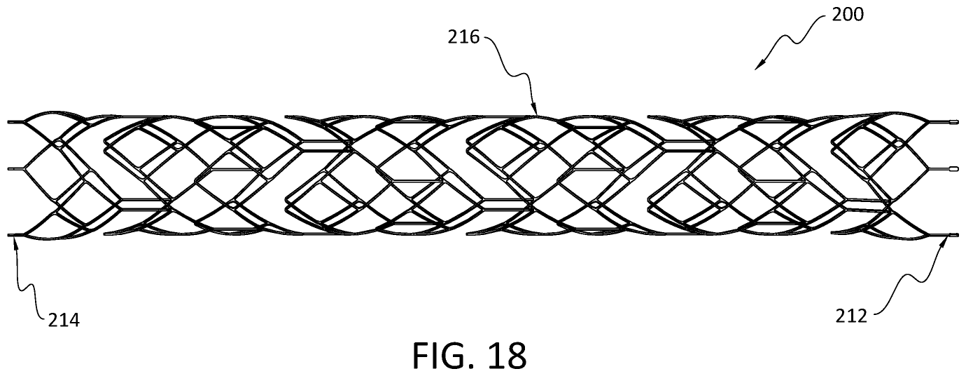

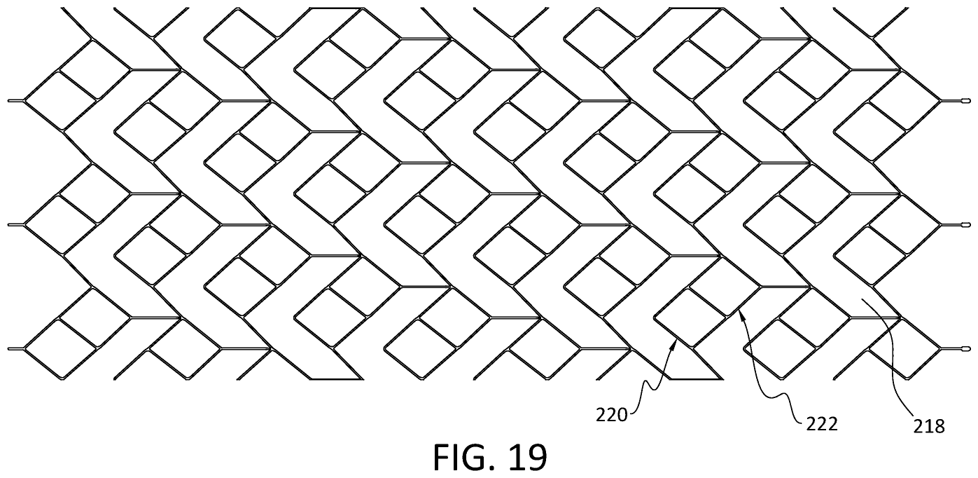

Referring now to FIG. 17-22, a fourth embodiment of a stent in accordance with the principles of the present invention is illustrated, designated generally as 200, in which a stent is provided that is resheathable as in the embodiment one and two (FIG. 1-10). As in the previous embodiments, the stent 200 has a proximal end 212 and a distal end 214 as oriented relative to the manner in which it is introduced. It includes a hybrid network cluster 216 of open cells 218, distal closed cells 220, and proximal closed cells 222. As will be disclosed below, the closed cells 220, 222 are connected in a configuration to be resheathable. The open cells 218 are not connected and the stent can be unsheathed to enhance flexibility.

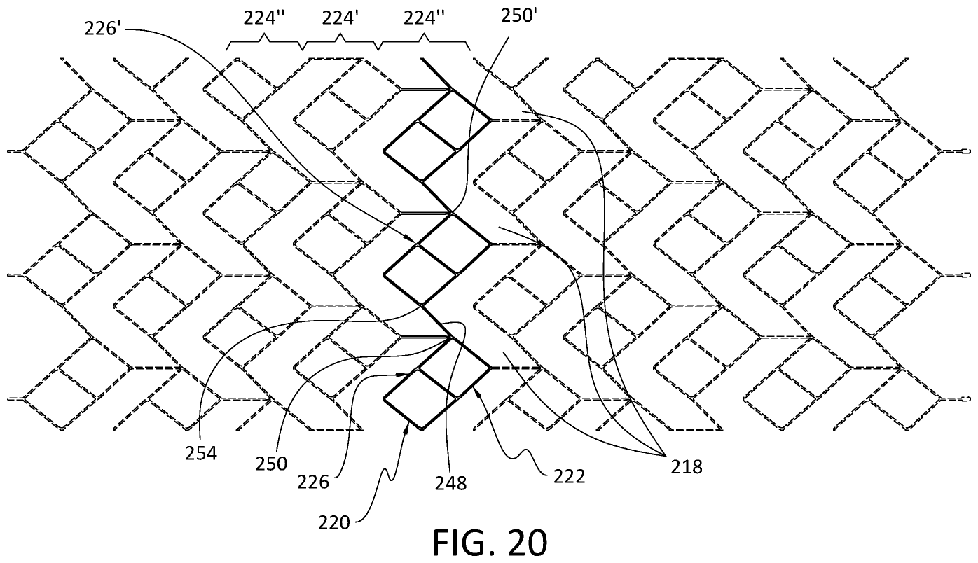

As can be best be seen in FIG. 20-22, the hybrid network cluster of open cells and closed cells comprises a plurality of rings 224, 224′, 224″ of pairs of the closed cells 220, 222. Each pair, designated generally as 226, includes a distal closed cell 220 and a proximal closed cell 222.

Each distal closed cell 220 has a substantially diamond-like shape. Each distal closed cell 220 includes a first distal cell strut 228, a second distal cell strut 230 opposing the first distal cell strut 228, a third distal cell strut 232 connecting the first distal cell strut 228 to the second distal cell strut 230 at a distal peak 234 at a distal end of the ring 224, and a shared strut 236 connecting the first distal cell strut 228 to the second distal cell strut 230.

The struts may have strut wall thicknesses in a range of 0.05-0.15 mm, preferably about 0.076 mm. The strut widths are approximately the same.

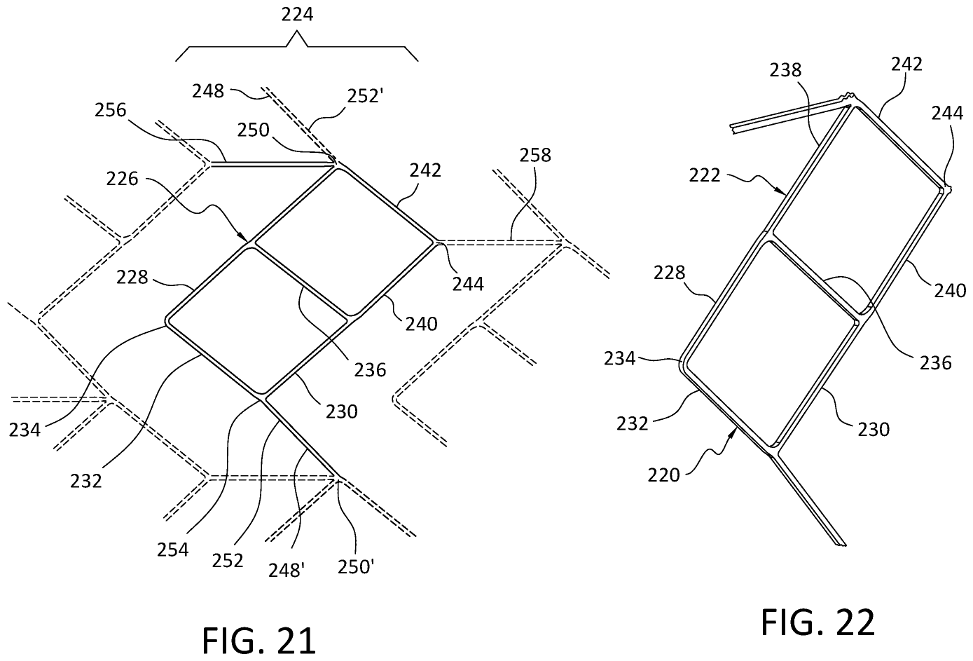

As best seen in FIGS. 21-22, each proximal closed cell 222 has a substantially diamond-like shape. Each proximal closed cell 222 includes a first proximal cell strut 238, a second proximal cell strut 240 opposing the first proximal cell strut 238, a third proximal cell strut 242 connecting the first proximal cell strut 238 to the second proximal cell strut 240 at a proximal peak 244 at a proximal end of the ring 224, and the shared strut 236 connecting the first proximal cell strut 238 to the second proximal cell strut 240.

Each ring 224 includes a plurality of straight connecting elements. Each straight connecting element connects a pair of closed cells to an adjacent pair of circumferentially spaced closed cells. A set of straight connecting elements is associated with each pair of said closed cells. With reference to pair 226 of closed cells, the set of straight connecting elements includes a first straight connecting element 248 extending from a first apex 250. The first apex 250 is a connection point of the first proximal cell strut 238 and the third proximal cell strut 242. A second straight connecting element 252 extends from a second apex 254. The second apex 254 is at a connection point of the second distal cell strut 230 and the third distal cell strut 232.

The first straight connecting element 248 functions as the second straight connecting element 252′ to a first apex 250′ of an adjacent second apex 254′ of an adjacent pair 226′ of circumferentially spaced closed cells.

Each ring includes a plurality of flexible connecting elements. Each flexible connecting element connects longitudinally adjacent rings. A set of flexible connecting elements is associated with each pair of the closed cells. With reference to pair 226 of closed cells, the set of flexible connecting elements includes a third straight connecting element 256 extending from the first apex 250. A fourth straight connecting element 258 extends from the proximal peak 244.

The first flexible connecting element 256 functions as a second flexible connecting element 258′ to a first apex 250′ of an adjacent pair 226′ of longitudinally spaced closed cells of an adjacent ring 224′. The first apex is positioned at a valley of the adjacent longitudinally spaced ring. As mentioned above, other embodiments and configurations may be devised without departing from the spirit of the invention and the scope of the appended claims.

1. A stent, comprising:
a hybrid network cluster of open cells and closed cells arranged wherein said closed cells are connected in a configuration to be resheathable; and,
wherein open cells are not connected and the stent can be unsheathed to enhance flexibility.

2. The stent of claim 1, said stent having a proximal end and a distal end, wherein said hybrid network cluster of open cells and closed cells comprises a plurality of rings of said closed cells, each ring of said closed cells, comprising:
a) a plurality of pairs of closed cells;
b) a plurality of straight connecting elements, each straight connecting element connecting a pair of closed cells to an adjacent pair of circumferentially spaced closed cells; and,
c) a plurality of flexible connecting elements, each flexible connecting element connecting longitudinally adjacent rings, wherein each pair of closed cells comprises a proximal peak at a proximal end and a distal peak at a distal end, said proximal peaks of a ring being connected by a flexible connecting element to a valley of an adjacent spaced ring,
wherein the distal peaks are free from constraint to enhance flexibility and wherein the proximal peaks are constrained for resheathability.

3. The stent of claim 1, said stent having a proximal end and a distal end, wherein said hybrid network cluster of open cells and closed cells comprises a plurality of rings of said closed cells, each ring of said closed cells, comprising:
a) a plurality of pairs of closed cells, each pair of closed cells, comprising:
i. a distal closed cell having a substantially diamond-like shape, including:
1. a first distal cell strut;
2. a second distal cell strut opposing said first distal cell strut;
3. a third distal cell strut connecting said first distal cell strut to said second distal cell strut at a distal peak at a distal end of the ring;
4. a shared strut connecting said first distal cell strut to said second distal cell strut;
ii. a proximal closed cell having a substantially diamond-like shape, including:
1. a first proximal cell strut;
2. a second proximal cell strut opposing to said first proximal cell strut;
3. a third proximal cell strut connecting said first proximal cell strut to said second proximal cell strut at a proximal peak at a proximal end of the ring;
4. said shared strut, being shared between said distal closed cell and said proximal closed cell, said shared strut connecting said first proximal cell strut to said second proximal cell strut;
b) a plurality of straight connecting elements, each straight connecting element connecting a pair of closed cells to an adjacent pair of circumferentially spaced closed cells, said plurality of straight connecting elements comprising a set of straight connecting elements associated with each pair of said closed cells, wherein each set of straight connecting elements, comprises:
i. a first straight connecting element extending from a first apex, said first apex being at a connection point of the first proximal cell strut and the third proximal cell strut;
ii. a second straight connecting element extending from a second apex, said second apex being at a connection point of the second distal cell strut and the third cell strut,
wherein said first straight connecting element functions as a second straight connecting element to a first apex of an adjacent second apex of an adjacent pair of circumferentially spaced closed cells;
c) a plurality of flexible connecting elements, each flexible connecting element connecting longitudinally adjacent rings, said plurality of flexible connecting elements comprising a set of flexible connecting elements associated with each pair of closed cells, wherein each set of flexible connecting elements, comprises:
i. a first flexible connecting element extending from said first apex; and,
ii. a second flexible connecting element extending from said proximal peak,
wherein said first flexible connecting element functions as a second flexible connecting element to a first apex of an adjacent pair of longitudinally spaced closed cells of an adjacent ring, wherein said first apex is positioned at a valley of the adjacent longitudinally spaced ring,
wherein the distal peaks are free from constraint to enhance flexibility and wherein the proximal peaks are constrained for resheathability.

4. The stent of claim 1, wherein said hybrid cluster is formed of a shape memory alloy (SMA).

5. The stent of claim 3, wherein said SMA comprises nitinol.

6. The stent of claim 3, wherein each ring of said plurality of rings comprises six pairs of closed cells.

7. The stent of claim 2, wherein each ring of said plurality of rings comprises three pairs of closed cells.

8. The stent of claim 3, wherein each ring of said plurality of rings comprises pairs of closed cells in a range of between two pairs and ten pairs.

9. The stent of claim 2, wherein each ring of said plurality of rings comprises six pairs of closed cells.

10. The stent of claim 1, wherein said hybrid network cluster of open cells and closed cells has a diameter in a range of 2 mm to 12 mm in a fully open position.

11. The stent of claim 1, wherein said hybrid network cluster of open cells and closed cells has a length in a range of 10 mm to 60 mm in a fully open position.

12. The stent of claim 2, wherein each flexible connecting element comprises an “S” configuration.

13. The stent of claim 2, wherein each flexible connecting element comprises a “V” configuration.

14. The stent of claim 1, further comprising a graft formed of expanded polytetrafluoroethylene (ePTFE) material positioned on an outer surface of the hybrid network cluster of open cells and closed cells.

15. The stent of claim 1, said stent having a proximal end and a distal end, wherein said hybrid network cluster of open cells and closed cells comprises a plurality of rings of said closed cells, each ring of said closed cells, comprising:
a) a plurality of closed cells;
b) a plurality of distally directed connecting elements, each distally directed connecting element connecting a closed cell of said plurality of closed cells to an adjacent circumferentially spaced closed cell via an associated distally directed connecting element of said adjacent circumferentially spaced closed cell;
c) a plurality of proximally directed connecting elements, each proximally directed connecting element connecting longitudinally adjacent rings, wherein each closed cell comprises a distal peak and a proximal peak, said proximal peak of said closed cell being connected by a proximally directed connecting element to a valley of an adjacent spaced ring; a first proximal ring being tapered; and,
d) a pushwire assembly positionable within an introducer sheath of a stent delivery system, said pushwire assembly having a proximal pushwire end and a distal pushwire end, said distal pushwire end being attached to proximal peaks of closed cells of said first proximal ring.

16. The stent of claim 15, wherein said first proximal ring is welded to said pushwire assembly.

17. The stent of claim 1, said stent having a proximal end and a distal end, wherein said hybrid network cluster of open cells and closed cells comprises a plurality of rings of said closed cells, each ring of said closed cells, comprising:
a) a plurality of closed cells, each closed cell, comprising:
i. a substantially diamond-like shape structure, including:
1. a first cell strut;
2. a second cell strut opposing said first cell strut;
3. a third cell strut connecting said first cell strut to said second cell strut at a distal peak at an end of the ring; and,
4. a fourth cell strut connecting said second cell strut to said first cell strut; and;
b) a plurality of first distally directed connecting elements, each first distally directed connecting elements connecting a distal apex of an open cell to an adjacent closed cell, said plurality of first distally directed connecting elements comprising a set of distally directed connecting elements associated with each of said closed cells, wherein each set of distally connecting elements, comprises:
i. a first distally directed connecting element extending from said distal apex of an open cell, said distal apex of an open cell being at a connection point of a second distally directed connecting element and said adjacent closed cell;
ii. a second distally directed connecting element extending from said distal apex of an open cell, said distal apex of an open cell being at a connection point of said first distally directed connecting element and said adjacent closed cell; and,
wherein said first distally directed connecting element functions as a second distally directed connecting element to a distal apex of an adjacent circumferentially spaced closed cell;
c) a plurality of proximally directed connecting elements, each proximally directed connecting element connecting longitudinally adjacent rings, said plurality of proximally directed connecting elements comprises a set of proximally directed connecting elements associated with each said closed cell and a valley of an adjacent ring wherein each set of proximally directed connecting elements, comprises:
i. a first proximally directed connecting element extending from a proximal peak of said closed cell; and,
ii. a second proximally directed connecting element extending from said valley;
wherein said first proximally directed connecting element functions as a second proximally directed connecting element to said valley of an adjacent ring, wherein said valley is positioned at an apex of an open cell of an adjacent longitudinally spaced ring,
wherein the distal peaks are free from constraint to enhance flexibility and wherein the proximal peaks are constrained for resheathability; and,
d) a pushwire assembly positionable within an introducer sheath of a stent delivery system, said pushwire assembly having a proximal pushwire end and a distal pushwire end, said distal pushwire end being attached to proximal peaks of closed cells of a first proximal ring.

18. The stent of claim 17 wherein said pushwire assembly comprises: a flexible laser cut hypotube formed of stainless steel with a core wire formed of Nitinol® alloy.

19. A stent delivery system for delivery and deploying an expandable stent, comprising:
a) a catheter;
b) a shaft comprising a distal end, disposed within the catheter; and,
c) an expandable stent comprising a proximal end and a distal end, wherein the shaft is coupled to the expandable stent and the shaft is disposed within the expandable stent, wherein the expandable stent comprises:
a hybrid network cluster of open cells and closed cells arranged wherein said closed cells are connected in a configuration to be resheathable; and,
wherein open cells are not connected and the stent can be unsheathed to enhance flexibility.

20. A method for deploying a resheathable stent for stent assisted coiling of hemorrhagic aneurysms and for treatment of intracranial atherosclerotic disease, comprising:
a) inserting a catheter into a vasculature of a patient, wherein a resheathable stent system is disposed with the catheter, the resheathable expandable stent comprising:
a hybrid network cluster of open cells and closed cells arranged wherein said closed cells are connected in a configuration to be resheathable; and,
wherein open cells are not connected and the stent can be unsheathed to enhance flexibility, and,
wherein longitudinal movement of a shaft relative to the resheathable expandable stent expands and contracts the resheathable stent.

21. A method for deploying a resheathable stent retriever for treatment of ischemic stroke to retrieve a blood clot in the vessels:
a) inserting a catheter into a vasculature of a patient, wherein a resheathable stent system is disposed with the catheter, the resheathable stent system device comprising:
a hybrid network cluster of open cells and closed cells arranged wherein said closed cells are connected in a configuration to be resheathable; and,
wherein open cells are not connected and the stent can be unsheathed to enhance flexibility, and,
wherein longitudinal movement of a shaft relative to the resheathable expandable stent expands and contracts the resheathable stent.

22. A retrievable stent, comprising:
a hybrid network cluster of open cells and closed cells arranged wherein said closed cells are connected in a configuration to be resheathable; and,
wherein open cells are not connected and the retrievable stent can be unsheathed to enhance flexibility; and, wherein a ring of proximal closed cells are tapered and the stent is retrievable.
